(19) United States
(12) Patent Application Publication
(10) Pub. No.: US 2016/0367503 A1
(43) Pub. Date: Dec. 22, 2016

(54) Combination Medication for Neuro-Degenerative Diseases

(71) Applicants: Cary Erwin Fechter, North Charleston, SC (US); Nancy Sharon Kedem, Melville, NY (US); Douglas F. Crane, Mt. Pleasant, SC (US); Thomas James Thomas, Summerville, SC (US)

(72) Inventors: Cary Erwin Fechter, North Charleston, SC (US); Nancy Sharon Kedem, Melville, NY (US); Douglas F. Crane, Mt. Pleasant, SC (US); Thomas James Thomas, Summerville, SC (US)

(21) Appl. No.: 14/745,400

(22) Filed: Jun. 20, 2015

(51) Int. Cl. A61K 31/145; A61K 31/445; A61K 31/137; A61K 31/135; A61K 31/343

(52) U.S. Cl. CPC A61K 31/145; A61K 31/135; A61K 31/343; A61K 31/137; A61K 31/445

(57) ABSTRACT

Presented are combination medications a wakefulness agent, an established Alzheimer's medication, and antidepressants. The combination serves several purposes. It will simplify medication delivery for both the patient and caregiver. Such a convenience will improve medication compliance.

The combination selects neuro-stimulants which individually improve cognition while reducing known drug side-effects.

The specific components are Modafinil, bupropion, SSRI's and Donepezil.

### TECHNICAL FIELD AND INDUSTRIAL APPLICABILITY OF THE INVENTION

This invention relates to the preparation of a pharmaceutical composition for the treatment of multiple symptoms of Alzheimer's disease and dementias. The present invention relates particularly to treating and improving excessive daytime sleepiness, depression, and memory loss in patients with mild to moderate Alzheimer's disease and dementias.

### BACKGROUND OF THE INVENTION

In 1906 Dr. Alois Alzheimer first recognized the disease now bearing his name. He described the disease as a dementia characterized by severe memory loss and confusion with pathological changes to neurons.

Frequency of diagnosis increased with the implementation of a cognitive measurement scale in 1968 and currently Alzheimer's disease is the most widely diagnosed form of dementia.

Understanding of the molecular basis of the disease improved with the discovery of the beta amyloid and tau proteins that form the toxic deposits causing neuron death. In spite of this, no curative agents have been developed although several acetylcholinesterase antagonists have been approved for treating memory deterioration. These drugs are currently being used with limited results.

More recently our research has revealed that two symptoms associated with Alzheimer's disease as well as other neurodegenerative diseases but not addressed by current therapeutic regimens, specifically depression and overall abnormal sleep patterns, can have a significant negative effect on the quality of life of patients. The abnormal sleep patterns in particular, by disrupting normal circadian rhythm, exacerbate the confusion, loss of mental focus, and feelings of disorientation characteristic of the disease. Furthermore, this abnormal circadian pattern of daytime sleepiness combined with restlessness and inability to sleep properly at night (sometimes referred to as “sundowning”) greatly complicates the efforts of caregivers.

Including treatment for these two symptoms in the overall treatment protocol can improve memory and patient quality of life as much as the drugs currently approved for memory deterioration.

Since traditional sleep regulating drugs such as tricyclics and benzodiazepines can increase the lethargy, confusion, and forgetfulness, already present in Alzheimer's patients, a different therapeutic approach is needed. Specifically, drugs of the benzhydrylsulfonylamide type can reduce excessive daytime sleepiness and promote normal circadian rhythm while avoiding the side effects of traditional sleep regulating drugs.

Combining this therapy with administration of a Selective Serotonin Reuptake Inhibitor (SSRI) antidepressant and, optionally, with a stimulating antidepressant such as bupropion reduces the severity of associated clinical depression symptoms. SSRI's also increase alpha secretase activity, which has been shown to aid in clearing amyloid beta, one of the peptides associated with formation of damaging plaques in the brain.

The overall results of this combination approach in conjunction with traditional acetylcholinesterase antagonist therapy are improvement in patient cognition including responsiveness and attitude toward their environment.

Additionally, combining all of the active pharmaceutical agents in a single formulation ensures that the timing of administration of the drugs is optimal. Since Alzheimer's caregivers typically have multiple duties to their patients, the single dosage format simplifies caregiver routine and improve compliance with the treatment regimen.

### EXAMPLE 1

A 50 year old female with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, and 5 mg. Donepezil once per day in the morning was initiated.
After six weeks the patient was re-evaluated by questionnaire using the ESS and found to be exhibiting a more normal sleep pattern. Patient responses demonstrated lessened depression criteria using BDS/DSM5.
Patient described overall attitude as more positive.

### EXAMPLE 2

A 65 year old female with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/DSM5.
A treatment protocol consisting of simultaneously administering 200 mg. Modafinil, 100 mg. Sertraline, and 5 mg. Donepezil once per day in the morning was initiated.
After six weeks the patient was re-evaluated by questionnaire through caregiver's responses using the ESS and found to be exhibiting a more normal sleep pattern. Caregiver responses demonstrated lessened patient depression criteria using BDS/DSM5.
Caregiver described patient's overall attitude as more positive.

### EXAMPLE 3

A 55 year old male with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, and 5 mg. Donepezil once per day in the morning was initiated.
After six weeks the patient was re-evaluated by questionnaire using the ESS and found to be exhibiting a more normal sleep pattern. Patient responses demonstrated lessened depression criteria using BDS/DSM5
Patient described overall attitude as more positive.

### EXAMPLE 4

A 71 year old male with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/DSM5.
A treatment protocol consisting of simultaneously administering 200 mg. Modafinil, 100 mg. Sertraline, and 5 mg. Donepezil once per day in the morning was initiated.
After six weeks the patient was re-evaluated by questionnaire through caregiver's responses using the ESS and found to be exhibiting a more normal sleep pattern. Caregiver responses demonstrated lessened patient depression criteria using BDS/DSM5.
Caregiver described patient's overall attitude as more positive.

### EXAMPLE 5

A 53 year old female with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, 5 mg. Donepezil, and 150 mg. Bupropion once per day in the morning was initiated.
After six weeks the patient was re-evaluated by questionnaire using the ESS and found to be exhibiting a more normal sleep pattern. Patient responses demonstrated lessened depression criteria using BDS/DSM5.
Patient described overall attitude as more positive.

### EXAMPLE 6

A 65 year old female with forgetfulness, previously diagnosed with moderate-severe Alzheimer's disease by a neurologist was evaluated through caregiver responses, using the ESS and found to be exhibiting excessive daytime sleepiness.

Caregiver responses to standard questionnaire provided positive indication of clinical depression using BDS/DSM5.
A treatment protocol consisting of simultaneously administering 200 mg. Modafinil, 100 mg. Sertraline, 5 mg. Donepezil, and 150 mg. Bupropion once per day in the morning was initiated.
After six weeks the patient was re-evaluated by questionnaire through caregiver's responses using the ESS and found to be exhibiting a more normal sleep pattern. Caregiver responses demonstrated lessened patient depression criteria using BDS/DSM5.
Caregiver described patient's overall attitude as more positive.

### EXAMPLE 7

A 50 year old male with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, 5 mg. Donepezil, and 150 mg. Bupropion once per day in the morning was initiated.
After six weeks the patient was re-evaluated by questionnaire using the ESS and found to be exhibiting a more normal sleep pattern. Patient responses demonstrated lessened depression criteria using BDS/DSM5.
Patient described overall attitude as more positive.

### EXAMPLE 8

A 72 year old male with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/DSM5.
A treatment protocol consisting of simultaneously administering 200 mg. Modafinil, 100 mg. Sertraline, 5 mg. Donepezil, and 150 mg. Bupropion once per day in the morning was initiated.
After six weeks the patient was re-evaluated by questionnaire through caregiver's responses using the ESS and found to be exhibiting a more normal sleep pattern. Caregiver responses demonstrated lessened patient depression criteria using BDS/DSM5.
Caregiver described patient's overall attitude as more positive.

What is claimed is:

1. A pharmaceutical composition comprising a therapeutically effective quantity of a wakefulness promoting stimulant of the formula: 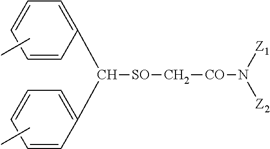
(Where either or both Z1 and Z2 may be hydrogen or C1-C4 alkyl; additionally Z1 may be Hydroxyl) combined with a therapeutically effective quantity of an SSRI antidepressant and suitable excipients.

2. A pharmaceutical composition as described in claim 1 containing a therapeutically effective quantity of a phenethylamine antidepressant in addition to the components of the composition of claim 1.

3. A pharmaceutical composition as described in claim 1 containing a therapeutically effective quantity of an acetylcholinesterase inhibitor in addition to the components of the composition of claim 1.

4. A pharmaceutical composition as described in claim 3 containing a therapeutically effective quantity of a stimulating antidepressant in addition to the components of the composition of claim 3.

5. A pharmaceutical composition according to claim 1 where the wakefulness promoting stimulant is modafinil (CAS No. 68693-11-8) and the SSRI antidepressant is sertraline (CAS No. 79617-96-2).

6. A pharmaceutical composition according to claim 1 where the wakefulness promoting stimulant is modafinil (CAS No. 68693-11-8) and the SSRI antidepressant is citralopram (CAS No. 59729-33-8).

7. A pharmaceutical composition according to claim 2 where the wakefulness promoting stimulant is modafinil (CAS No.68693-11-8), the SSRI antidepressant is sertraline (CAS No. 79617-96-2), and the phenethylamine antidepressant is bupropion (CAS No. 34841-39-9).

8. A pharmaceutical composition according to claim 2 where the wakefulness promoting stimulant is modafinil (CAS No. 68693-11-8), the SSRI antidepressant is citralopram (CAS No. 59729-33-8), and the phenethylamine antidepressant is bupropion (CAS No. 34841-39-9).

9. A pharmaceutical composition according to claim 3 where the wakefulness promoting stimulant is modafinil (CAS No. 68693-11-8), the SSRI antidepressant is sertraline (CAS No. 79617-96-2), and the acetylcholinesterase inhibitor is donepezil (CAS No. 120014-06-4)

10. A pharmaceutical composition according to claim 3 where the wakefulness promoting stimulant is modafinil (CAS No. 68693-11-8), the SSRI antidepressant is citralopram (CAS No. 59729-33-8), and the acetylcholinesterase inhibitor is donepezil (CAS No. 120014-06-4)

11. A pharmaceutical composition according to claim 4 where the wakefulness promoting stimulant is modafinil (CAS No. 68693-11-8), the SSRI antidepressant is sertraline (CAS No. 79617-96-2 citralopram (CAS No. 59729-33-8), the acetylcholinesterase inhibitor is donepezil (CAS No. 120014-06-4), and the phenethylamine antidepressant is bupropion (CAS No. 34841-39-9).

12. A pharmaceutical composition according to claim 4 where the wakefulness promoting stimulant is modafinil (CAS No. 68693-11-8), the SSRI antidepressant is citralopram (CAS No. 59729-33-8), the acetylcholinesterase inhibitor is donepezil (CAS No. 120014-06-4), and the phenethylamine antidepressant is bupropion (CAS No. 34841-39-9).

13. A pharmaceutical composition according to claim 5 where the quantity of Modafinil (CAS No. 68693-11-8) is between 50 and 500 milligrams and the quantity of sertraline (CAS No. 79617-96-2) is between 15 and 250 milligrams.

14. A pharmaceutical composition according to claim 6 where the quantity of modafinil (CAS No. 68693-11-8) is between 50 and 500 milligrams and the quantity of citralopram (CAS No. 59729-33-8) is between 5 and 50 milligrams.

15. A pharmaceutical composition according to claim 7 where the quantity of modafinil (CAS No. 68693-11-8) is between 50 and 500 milligrams, the quantity of sertraline (CAS No. 79617-96-2) is between 15 and 250 milligrams, and the quantity of bupropion (CAS No. 34841-39-9) is between 50 and 400 milligrams.

16. A pharmaceutical composition according to claim 8 where the quantity of modafinil (CAS No. 68693-11-8) is between 50 and 500 milligrams, the quantity of citralopram (CAS No. 59729-33-8) is between 5 and 50 milligrams, and the quantity of bupropion (CAS No. 34841-39-9) is between 50 and 400 milligrams.

17. A pharmaceutical composition according to claim 9 where the quantity of modafinil (CAS No. 68693-11-8) is between 50 and 500 milligrams, the quantity of sertraline (CAS No. 79617-96-2) is between 15 and 250 milligrams, and the quantity of donepezil (CAS No. 120014-06-4) is between 1.5 and 12.5 milligrams.

18. A pharmaceutical composition according to claim 10 where the quantity of modafinil (CAS No. 68693-11-8) is between 50 and 500 milligrams, the quantity of citralopram (CAS No. 59729-33-8) is between 5 and 50 milligrams, and the quantity of donepezil (CAS No. 120014-06-4) is between 1.5and 12.5 milligrams.

19. A pharmaceutical composition according to claim 11 where the quantity of modafinil (CAS No. 68693-11-8) is between 50 and 500 milligrams, the quantity of sertraline (CAS No. 79617-96-2) is between 15 and 250 milligrams, the quantity of bupropion (CAS No. 34841-39-9) is between 50 and 400 milligrams, and the quantity of donepezil (CAS No. 120014-06-4) is between 1.5 and 12.5 milligrams.

20. A pharmaceutical composition according to claim 12 where the quantity of modafinil (CAS No. 68693-11-8) is between 50 and 500 milligrams, the quantity of citralopram (CAS No. 59729-33-8) is between 5 and 50 milligrams, the quantity of bupropion (CAS No. 34841-39-9) is between 50 and 400 milligrams, and the quantity of donepezil (CAS No. 120014-06-4) is between 1.5 and 12.5 milligrams.

21. A pharmaceutical composition comprising a therapeutically effective quantity of a wakefulness promoting stimulant of the formula: 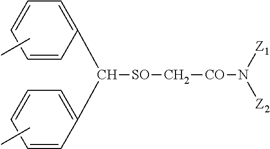
(Where either or both Z1 and Z2 may be hydrogen or C1-C4 alkyl; additionally Z1 may be hydroxyl).
combined with a therapeutically effective quantity of a stimulating antidepressant and suitable excipients.

22. A pharmaceutical composition as described in claim 21 containing a therapeutically effective quantity of an acetylcholinesterase inhibitor in addition to the components of the composition of claim 21.

23. A pharmaceutical composition according to claim 21 where the wakefulness promoting stimulant is modafinil (CAS No: 68693-11-8) and the stimulating antidepressant is bupropion (CAS No. 34841-39-9).

24. A pharmaceutical composition according to claim 22 where the wakefulness promoting stimulant is Modafinil (CAS No. 68693-11-8), the acetylcholinesterase inhibitor is donepezil (CAS No. 120014-06-4), and the stimulating antidepressant is bupropion (CAS No. 34841-39-9).

25. A pharmaceutical composition according to claim 23 where the quantity of modafinil (CAS No. 68693-11-8) is between 50 and 500 milligrams and the quantity of bupropion (CAS No. 34841-39-9) is between 50 and 400 milligrams.

26. A pharmaceutical composition according to claim 24 where the quantity of modafinil (CAS No. 68693-11-8) is between 50 and 500 milligrams, the quantity of bupropion (CAS No. 34841-39-9) is between 50 and 400 milligrams, and the quantity of donepezil (CAS No. 120014-06-4) is between 1.5 and 12.5 milligrams.

27. A therapeutic protocol for treating multiple symptoms of Alzheimer's syndrome by administering the pharmaceutical compositions described in claims 1-26.
